(19) United States
(12) Patent Application Publication
(10) Pub. No.: US 2015/0366551 A1
(43) Pub. Date: Dec. 24, 2015

(54) PATIENT-SUPPORTED OPHTHALMIC PLATFORMS

(71) Applicant: The Board of Trustees of The Leland Stanford Junior University, Palo Alto, CA (US)

(72) Inventors: Mark BLUMENKRANZ, Palo Alto, CA (US); Kenneth N. HORNE, San Francisco, CA (US); Kinman HONG, Cupertino, CA (US); Naveen JAYAKUMAR, Cupertino, CA (US)

(73) Assignee: The Board of Trustees of The Leland Stanford Junior University, Palo Alto, CA (US)

(21) Appl. No.: 14/843,568

(22) Filed: Sep. 2, 2015

Related U.S. Application Data
(63) Continuation of application No. PCT/US2014/023229, filed on Mar. 11, 2014.
(60) Provisional application No. 61/778,800, filed on Mar. 13, 2013.

(51) Int. Cl. A61B 17/02; A61M 16/06; A61F 9/04

(52) U.S. Cl. CPC A61B 17/0231; A61F 9/04; A61M 16/06; A61G 13/121

(57) ABSTRACT

Patient-supported ophthalmic platforms for use in ophthalmic procedures are described. The platform is supported at least in part upon a patient's face where the platform is configured to cover at least one or both eyes and further includes a conformable portion which rests upon a remainder of the patient's face. The platform may also integrate rests for the hands of the surgeon and may optionally integrate an instrument rest for allowing ready access of one or more surgical instruments to the surgeon. One or more corresponding eyelid speculums may also be integrated with the surgical platform eliminating the need for a separate speculum. An irrigation fluid trough may also be integrated with the surgical platform for collecting and diverting an irrigating fluid and the platform may optionally be used in combination with an oxygen delivery mask or nasal cannula.

### CROSS-REFERENCE TO RELATED APPLICATIONS

This application is a continuation of International Application No. PCT/US2014/023229 filed Mar. 11, 2014 which claims the benefit of priority to U.S. Provisional Application No. 61/778,800 filed Mar. 13, 2013, which are incorporated herein by reference in their entirety.

### FIELD OF THE INVENTION

The present invention relates generally to medical devices and methods for facilitating and performing ophthalmic surgical procedures. More particularly, the present invention relates to surgical platform apparatus and methods of use which are supported at least in part by the patient for facilitating ophthalmic surgical procedures.

### BACKGROUND OF THE INVENTION

Generally, ophthalmic surgical procedures require draping of the patient and of the surrounding areas with a sterile barrier to create and maintain a sterile field. The purpose of draping is to eliminate the passage of microorganisms between the non-sterile environment and the sterile surgical theater. Draping materials may be disposable or non-disposable and are generally made of absorbent or non-absorbent paper or plastic materials or a combination of both. A sterile drape is typically also applied to the face and surrounding eyelashes and eyelids to help protect the operative eye field away from potentially contaminated surfaces. Additionally, an eyelid speculum separate from the draping may also be used to help improve the exposure of the surgical field in or around one or both eyes.

However, the draping process may take up to several minutes and often requires the assistance of several sterile assistants. Moreover, because the drapes used are variable and can incorporate a large split sheet covering the patient's chest and head, an additional eyelid ophthalmic drape is often placed over the split sheet and requires the surgeon to manually cut an opening through the eyelid drape to conform it across the eyelid margins as an adhesive. The separate eyelid speculum, usually a non-disposable device, is usually placed to gain appropriate exposure to the operative ocular field.

Not only is the draping process inconvenient and time consuming, additional procedures such as placing an eyelid speculum to expose the operative field and a nasal cannula to provide supplemental oxygen to the patient take time. Such a process represents an inconvenience to the surgeons due to the prolonged time-consuming surgical set-up time and assistance of several staff members. Moreover, several different costly instruments are required in addition to the drapes such as the eyelid speculums, scissors for perforating the eyelid drape, nasal cannulas, etc. Also, the set-up may be uncomfortable to the patients due to the draping process and placement of the nasal cannula which often create irritation around the nose and the ears.

Thus, there is a need for improving the efficiency in surgical set-up time as well as for improving the efficiency during surgical procedures by providing an integrated surgical draping system which also provides a cost savings. There is also a need for providing an integrated eyelid speculum that maintains optimum exposure while relieving the globe of any posterior pressure.

### SUMMARY OF THE INVENTION

A surgical platform for use in ophthalmic procedures may be supported at least in part upon a portion of a patient's face where the platform is configured to cover at least one or both eyes of the patient and further includes a conformable portion which rests upon a remainder of the patient's face. The platform may also integrate rests for the hands of the surgeon and may optionally integrate an instrument rest for allowing ready access of one or more surgical instruments to the surgeon.

One or more corresponding eyelid speculums may also be integrated with the surgical platform eliminating the need for a separate speculum. An irrigation fluid trough may also be integrated with the surgical platform for collecting and diverting any irrigating fluid and the platform may optionally be used in combination with an oxygen delivery mask or nasal cannula.

In another variation, the surgical assembly may generally comprise a support frame which is positionable upon a face of a patient, the support frame having at least a first supporting rim in proximity to at least one eye of the patient, at least one speculum adjustably attached to the first supporting rim such that the at least one speculum is configured to retract at least an upper or lower eyelid of the patient, a platform attached to the support frame and extending over at least a first part of the face of the patient, and a conformable portion attached to the support frame adjacent to the platform and extending over a second part of the face of the patient.

Another variation may generally comprise a headband adjustably securable over a head of a patient, a platform attachable to the a portion of the headband, at least one speculum adjustably attached to a second portion of the headband, where the at least one speculum extends from the second portion into proximity to at least one eye of the patient, and an irrigation trough fluidly coupled to the headband.

In yet another variation, the assembly may generally comprise a first block having a first contoured surface configured for placement against a first side of a patient's head, a second block having a second contoured surface configured for placement against a second side of the patient's head, and a conformable shield extending between the first block and second block for placement over a face of the patient's head, wherein the conformable shield defines at least one opening for alignment over at least one eye to be treated.

In one example, the ophthalmic surgical assembly may generally comprise a platform having a flattened surface to provide for an instrument rest and which is also configured to conform over a portion of the face of the patient. The platform may extend at least partially over the patient's face to cover one or both eyes. The platform may also define a contoured edge which extends around and provides a border relative to the patient's remaining eye, nose, and mouth. Optionally, a conformable portion may be attached to a portion of the contoured edge and extend over the eye to be treated as well as over a remaining portion of the patient's face.

While the platform may be comprised of any number of polymers, plastics, metals, or combination thereof which are biocompatible and which provide a relative stiffened platform, the conformable portion may be formed from a conformable polymer, such as silicone, which may extend over the patient's face and conform in part to the underlying features while further draping over a side of the patient's face where it may optionally form an irrigation trough. The irrigation trough may be formed along a distal edge of the conformable portion integrally or separately to collect any irrigating fluid and/or debris which may run off from the surgical field.

The conformable portion of the surgical assembly may be laid atop the eye to be treated such that an opening defined through the conformable portion is aligned with the eye of interest while the remainder of the patient's face is covered or draped by the portion. The opening may be aligned with the patient's eye to provide surgical access to the eye while allowing for the remainder of the patient's face to be covered or draped. Because the opening provides for a defined surgical field or theater for the eye to be treated, one or more adjustable eyelid speculums may be integrated or otherwise attached to the conformable portion in proximity to the opening such that the speculums may retract the eyelids of the patient to provide for a clear surgical field. With the speculums supported by the conformable portion pressure on the underlying surface of the eye may be alleviated by the weight of the speculums and retracted eyelids being supported by the conformable portion.

While the surgical assembly may simply rest upon the face of the patient during a procedure, the platform and/or the conformable portion may be optionally adhered temporarily to the patient's face to inhibit or prevent movement of the assembly. Alternatively and/or additionally, the surgical assembly may incorporate an adjustable band which may be used to secure the assembly to the patient's head.

In another variation, an additional hand rest or stabilizer may be positioned along an edge of the platform overlying the patient's forehead. The hand rest or stabilizer may comprise an elevated platform or cushion to provide an area where the surgeon may rest their hands during a procedure upon the eye (or eyes) or an area where the surgeon may use to position or reposition the patient's head.

In yet another variation, the surgical assembly may be configured to be supported and/or secured by the patient using a supporting scaffold or frame placed over or upon the patient's face and/or head. A supporting frame in the form of, e.g., eyeglasses, may be placed upon the face of the patient such that the supporting frame extends towards the patient's ears and has at least a first supporting rim and optional second supporting rim which may be positioned over the respective eyes of the patient. A platform may be attached or otherwise coupled to the supporting frame to provide an instrument rest such that the platform extends from a first upper portion of the frame to rest above the patient's forehead. A conformable portion may be attached to a second lower portion of the supporting frame to extend over and rest upon the patient's face. The distal edge of the conformable portion may define a contoured edge to accommodate the patient's nose and mouth and which may also incorporate an optional irrigation trough. One or both supporting rims may also incorporate one or more eyelid speculums to provide a clear surgical field to the underlying eye (or eyes) to be treated. Alternatively, one or both of the supporting rims may incorporate a covering to cover the underlying eye (or eyes) partially or completely.

In yet another variation, a surgical platform may be attached or coupled via an attachment to the supporting frame which may rest upon the patient's face. The platform may be positioned along the supporting frame over where first or second supporting rim would normally reside such that the underlying eye is covered. The remaining eye to be treated may have its eyelids retracted by the corresponding speculum adjustably integrated along the supported rim. An irrigation trough may also be integrated with the conformable portion or optionally integrated with the support frame itself as shown.

Yet another variation of a supporting frame may incorporate an optional light source having an adjustable arm attached to the supporting frame, e.g., via attachment. The light source may be attached, for instance, along second supporting rim and extend via an adjustable arm such that its distal end is in proximity to the first supporting rim to provide additional lighting to the underlying eye to be treated. Additionally and/or alternatively, the light source may incorporate a fluid lumen through which irrigation fluid, such as saline, may be flowed to provide a clearing fluid in addition to lighting to the treated eye.

In yet another variation, an ophthalmic surgical block assembly may be positioned along either side of the patient's head to provide support and stabilization rather than being positioned to rest directly upon the patient's face. The surgical block assembly may generally comprise a first block and a second block which may be positioned along both sides of the patient's head. The first block may include a first backing with a first conformable rest which may be fabricated from any number of materials suitable for cushioning against the patient's head, such as foam. The conformable rest may define a first contoured surface which may be sized for placement against a side of the patient's head. Likewise, the second block may include a second backing and a second conformable rest which defines a second contoured surface for placement and cushioning against the other side of the patient's head in apposition to the first block.

With the patient's head secured between the first and second block, a conformable polymer shield may be positioned over the patient's face to provide for draping of the patient. The shield may be attached to the first and second block via respective first and second attachment and may further define an opening through which the patient's face may be accessed. Alternatively, portions of the shield may define one or more openings to accommodate one or both eyes for treatment as well as openings to accommodate the patient's nose and/or mouth. The size of each block may be optionally adjustable in height as well as relative spacing from one another and from the patient's head to accommodate varying head sizes, if so desired. Another variation may include an optional oxygen mask incorporated for placement over the patient's mouth and nose as well as a separate instrument rest which may be positioned along one of both of the blocks.

Yet another variation may include a surgical platform which may be placed around a patient's face. The platform may provide a relatively broader surface upon which one or more instrument rests may be provided. The platform may be supported upon the patient's face or head and retained in position via a headband attached via one or more headband attachments on either side of the platform. The opening of the platform may be varied in diameter as well for allowing for adjustability of the platform relative to the patient's head. Moreover, the platform may define one or more openings suitably sized for placement around a periphery of the patient's face in which case a conformable polymer shield may be placed over the patient's face with one or more openings for providing access to one or both eyes for treatment.

### BRIEF DESCRIPTION OF THE DRAWINGS

FIGS. 1A to 1C show front, perspective, and end views, respectively, of one variation of an ophthalmic surgical platform which may rest upon a portion of the patient's face and may further comprise a conformable portion.

FIG. 2 shows a perspective view of another variation which integrates a hand rest for the practitioner as well as an oxygen mask.

FIGS. 3A to 3C show front, perspective, and side views, respectively, of another variation of an ophthalmic surgical platform which may rest around the patient's face and may further comprise a conformable portion which rests over the patient's face.

FIG. 4 shows a perspective view of the embodiment of FIG. 3A which also integrates an oxygen mask.

FIGS. 5A and 5B show front and perspective views of another variation of a surgical platform integrated with and supported by a frame or scaffold formed as an eyeglass-like support structure.

FIG. 6 shows a perspective view of another variation of a surgical platform supported by an eyeglass support structure also having a conformable portion and an oxygen mask.

FIG. 7 shows a perspective view of yet another variation of a surgical platform supported by an eyeglass support structure having an integrated trough, oxygen mask, and instrument rest positioned over an eye.

FIGS. 8A and 8B show front and perspective views of another variation of an eyeglass support structure having an integrated light and/or irrigation assembly.

FIG. 9A shows a side view of one variation of the relative positioning of the integrated light and/or irrigation assembly.

FIG. 9B shows a perspective detail view of an eyelid speculum integrated into the eyeglass support structure.

FIGS. 10A and 10B show perspective views of another variation of an ophthalmic surgical platform supported by a headband assembly.

FIGS. 11A to 11C show perspective, end, and detail perspective views of yet another variation of a surgical blocking assembly for stabilizing a patient's head and face.

FIG. 12 shows a perspective view of another variation of a surgical blocking assembly having an integrated instrument platform and a conformable portion.

FIG. 13 shows a perspective view of another variation of a surgical platform which may reside around the patient's face.

FIGS. 14A and 14B show perspective views of examples of the relative positioning of the surgical platform of FIG. 13 with respect to the patient's face.

### DETAILED DESCRIPTION OF THE INVENTION

An ophthalmic surgical platform may be comprised generally of a platform which is supported at least in part upon a portion of a patient's face. The platform itself may be shaped to cover at least one or both eyes of the patient and may further include a conformable portion which may rest upon a remainder of the patient's face. The portion of the platform which rests upon the patient's face may also integrate rests for the hands of the surgeon during a procedure or the portion may optionally integrate an instrument rest for allowing ready access of one or more surgical instruments to the surgeon.

Additionally, one or more corresponding eyelid speculums may also be integrated with the surgical platform eliminating the need for a separate speculum. An irrigation fluid trough may also he integrated with the surgical platform for collecting and diverting any irrigating fluid used during a procedure. Furthermore, the platform may optionally be used in combination with an oxygen delivery mask or nasal cannula as well. The platform may be reusable between different patients or it may be configured to be disposable after a single use or several uses.

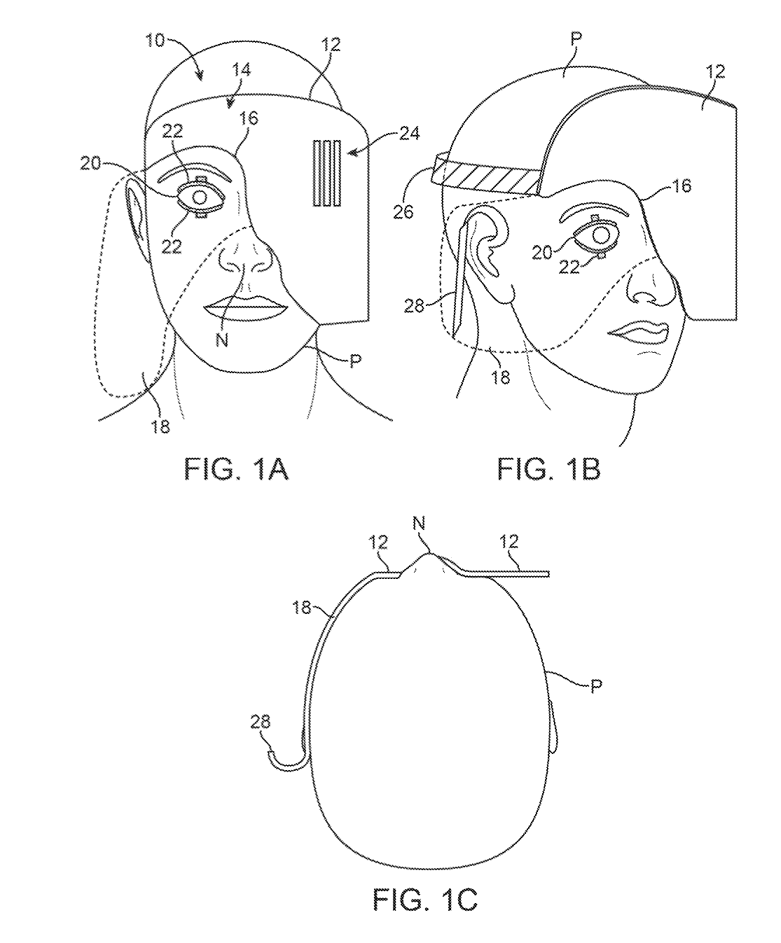

One variation is illustrated in the front and perspective views of FIGS. 1A and 1B which show an ophthalmic surgical assembly 10 generally comprising a platform 12 which may have a flattened surface 14 to provide for an instrument rest 24 and which is also configured to conform over a portion of the face of the patient P. The platform 12 may extend at least partially over the patient's face to cover one or both eyes although shown in this example as covering a single eye. The platform 12 may also define a contoured edge 16 which extends around and provides a border relative to the patient's remaining eye, nose N, and mouth. Optionally, a conformable portion 18 may be attached to a portion of the contoured edge 16 and extend over the eye to be treated as well as over a remaining portion of the patient's face.

While the platform 12 may be comprised of any number of polymers, plastics, metals, or combination thereof which are biocompatible and which provide a relative stiffened platform, the conformable portion 18 may be formed from a conformable polymer, such as silicone, which may extend over the patient's face and conform in part to the underlying features while further draping over a side of the patient's face where it may optionally form an irrigation trough 28, as shown in the end view of FIG. 1C, which illustrates the platform 12 and conformable portion 18 placed upon the face of the patient P. The irrigation trough 28 may be formed along a distal edge of the conformable portion 18 integrally or separately to collect any irrigating fluid and/or debris which may run off from the surgical field. The variation shown illustrates a variation where the patient's nose N and mouth may be left exposed to accommodate other instruments or accessories such as nasal cannulas, oxygen masks, endotrachial tubes, etc.

The conformable portion 18 of the surgical assembly 10 may be laid atop the eye to be treated such that an opening 20 defined through the conformable portion 18 is aligned with the eye of interest while the remainder of the patient's face is covered or draped by the portion 18. The opening 20 may be aligned with the patient's eye to provide surgical access to the eye while allowing for the remainder of the patient's face to be covered or draped. Because the opening 20 provides for a defined surgical field or theater for the eye to be treated, one or more adjustable eyelid speculums 22 may be integrated or otherwise attached to the conformable portion 18 in proximity to the opening 20 such that the speculums 22 may retract the eyelids of the patient to provide for a clear surgical field. With the speculums 22 supported by the conformable portion 18 pressure on the underlying surface of the eye may be alleviated by the weight of the speculums 22 and retracted eyelids being supported by the conformable portion 18.

While the surgical assembly 10 may simply rest upon the face of the patient P during a procedure, the platform 12 and/or the conformable portion 18 may be optionally adhered temporarily to the patient's face to inhibit or prevent movement of the assembly 10. Alternatively, the surgical assembly 10 may incorporate an adjustable band 26 which may be used to secure the assembly 10 to the patient's head, as shown. While the surgical assembly 10 may be standardized in size, e.g., one or more standard sizes for adults and children, the assembly 10 shown, as well as in other variations described herein, may also be custom-fitted to a particular patient's anatomy to ensure a conforming fit to the patient's face.

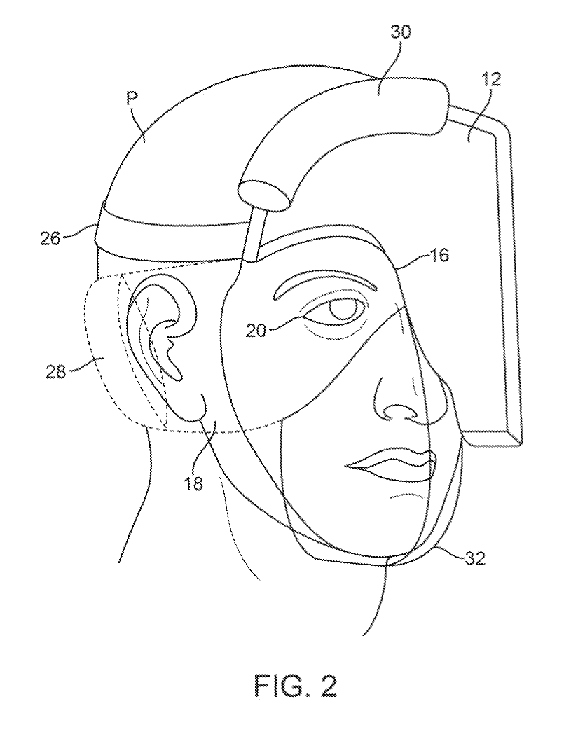

In another variation, FIG. 2 shows a perspective view of a platform 12 defining the contoured edge 16 and conformable portion 18 extending over the eye to be treated. An optional oxygen mask 32 is also illustrated secured over the patient's nose and mouth adjacent to the conformable portion 18 and platform 12. An additional hand rest or stabilizer 30 is also shown positioned along an edge of the platform overlying the patient's forehead. The hand rest or stabilizer 30 may comprise an elevated platform or cushion to provide an area where the surgeon may rest their hands during a procedure upon the eye (or eyes) or an area where the surgeon may use to position or reposition the patient's head.

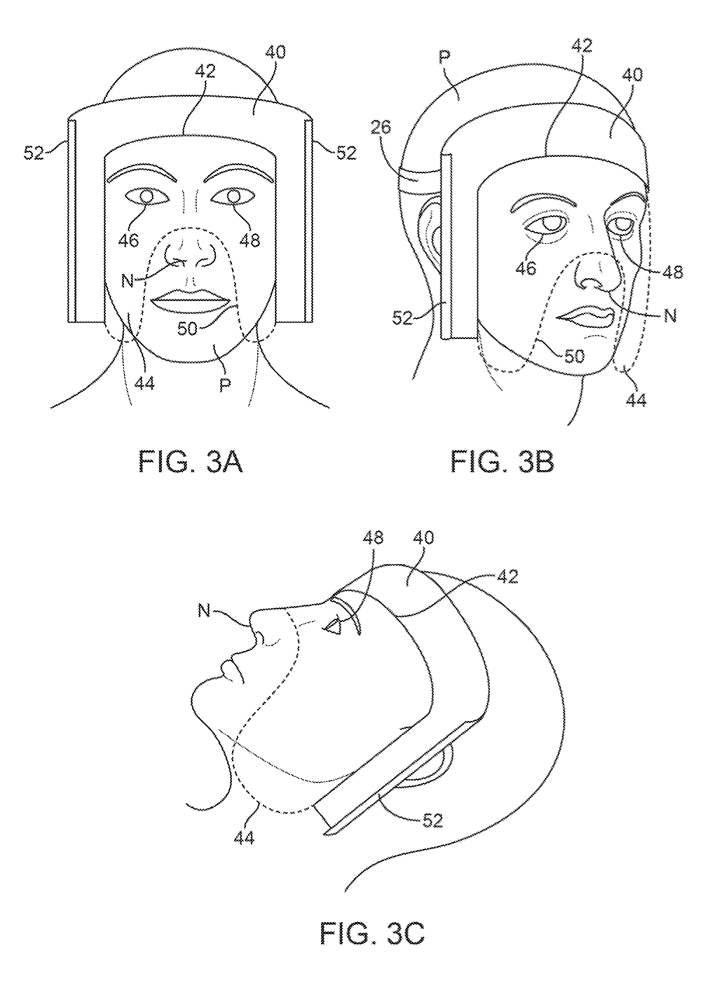

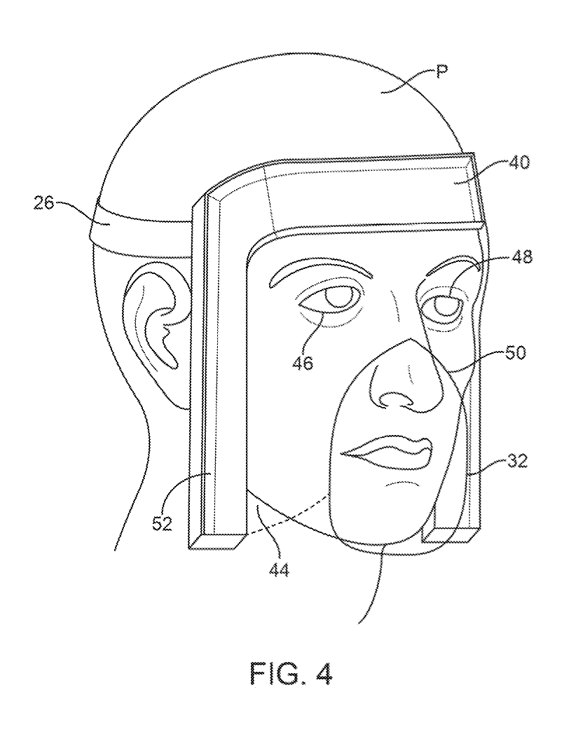

FIGS. 3A to 3C show front, perspective, and side views, respectively, of yet another variation where the platform 40 may be shaped to be positioned around a periphery of the patient's face such that the platform 40 is formed to lie over the patient's forehead and extend along the sides of the patient's face. The contoured edge 42 formed by the platform 40 may frame the patient's face between two portions which may integrate one or more irrigation troughs 52. A conformable portion 44 may extend between the contoured edges 42 of the platform 40 and may further define a first and/or second opening 46, 48 which may overlie one or both respective eyes to be treated. As described above, the first and/or second opening 46, 48 may further incorporate respective eyelid speculums. Additionally, a lower edge of the conformable portion 44 may further define a contoured edge 50 which may extend around the patient's nose N and mouth to accommodate additional instruments or accessories, as described above. The remainder of the contoured edge 50 may extend distally from the contoured edge 42 of the platform 40 to cover the remainder of the patient's face. FIG. 4 shows a perspective view of the platform 40 which integrates the irrigation trough 52. Also shown are the conformable portion 44 extending over the patient's face along with the oxygen mask 32 positioned over the patient's nose and mouth adjacent to the contoured edge 50. In this example, the platform 40 may also incorporate a raised edge or lip around a periphery of the platform 40 and trough 52 to retain any irrigation fluid or debris within the platform 40 as well as an optional headband 26 for securing the platform 40 to the patient's head.

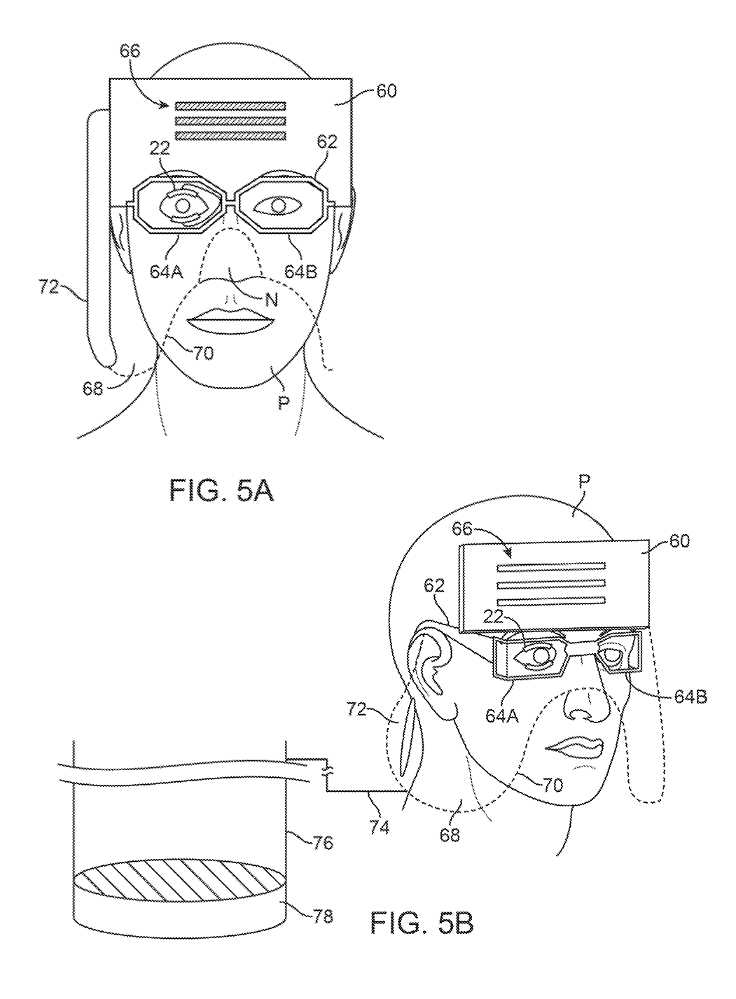

In yet another variation, the surgical assembly may be configured to be supported and/or secured by the patient using a supporting scaffold or frame placed over or upon the patient's face and/or head. In the embodiment shown in FIG. 5A, a front view of a supporting frame 62 in the form of e.g., eyeglasses, may be placed upon the face of the patient P such that the supporting frame 62 extends towards the patient's ears and has at least a first supporting rim 64A and optional second supporting rim 64B which may be positioned over the respective eyes of the patient. A platform 60 may be attached or otherwise coupled to the supporting frame 62 to provide an instrument rest 66 such that the platform 60 extends from a first upper portion of the frame 62 to rest above the patient's forehead. A conformable portion 68 may be attached to a second lower portion of the supporting frame 62 to extend over and rest upon the patient's face, as described above. The distal edge of the conformable portion 68 may define a contoured edge 70 to accommodate the patient's nose and mouth and which may also incorporate an optional irrigation trough 72. One or both supporting rims 64A, 64B may also incorporate one or more eyelid speculums 22 to provide a clear surgical field to the underlying eye (or eyes) to be treated. Alternatively, one or both of the supporting rims 64A, 64B may incorporate a covering to cover the underlying eye (or eyes) partially or completely.

In this variation (or in other variations described herein), a fluid collection reservoir 76 may be optionally coupled via a fluid line 74 to the one or more troughs 72, as shown in the perspective view of FIG. 5B. The reservoir 76 may be fluidly coupled to collect and retain any irrigation fluid 78 or debris which may be cleared from the patient during a procedure and collected in the trough 72.

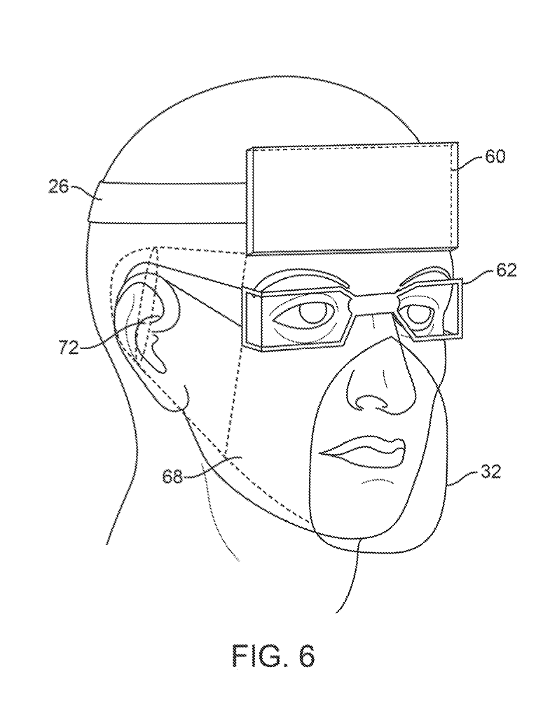

Another variation is shown in the perspective view of FIG. 6 which illustrates a platform 60 adjustably secured to the patient's head via headband 26. An example of the supporting frame 62 is shown secured upon the patient's face and behind the ears (like eyeglasses) and the conformable portion 68 is shown attached to the supporting frame 62 and positioned upon the patient's face. An optional oxygen mask 32 is shown placed over the patient's nose and mouth adjacent to the conformable portion 68. In this and other variations, the oxygen mask 32 may be optionally attached or coupled to the conformable portion 68 such that the assembly may be placed and positioned upon the patient's face as an integrated assembly.

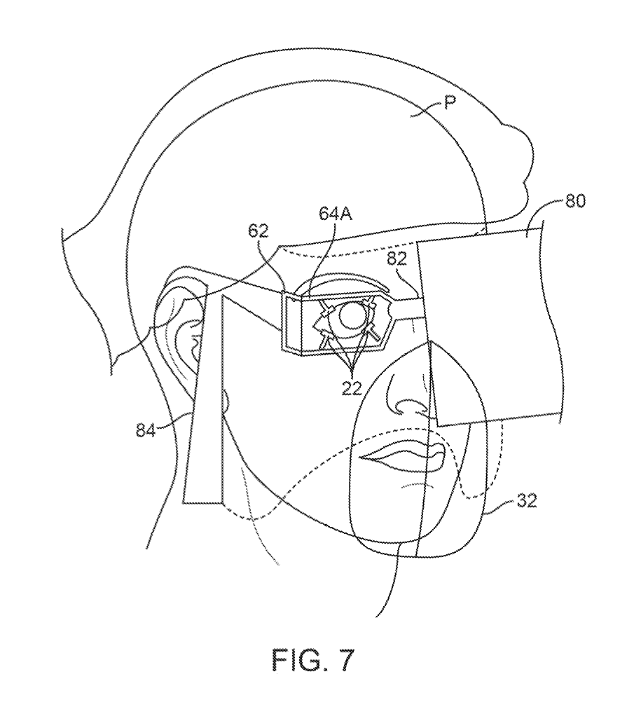

In yet another variation, FIG. 7 shows a perspective view of a surgical platform 80 attached or coupled via an attachment 82 to the supporting frame 62 which may rest upon the patient's face. The platform 80 may be positioned along the supporting frame 62 over where first or second supporting rim 64B would normally reside such that the underlying eye is covered. The remaining eye to be treated may have its eyelids retracted by the corresponding speculum 22 adjustably integrated along the supported rim 64A. The conformable portion 68 may also be seen overlying the remainder of the patient's face with an oxygen mask 32 optionally integrated with the conformable portion 68 and/or supporting frame 62. An irrigation trough 84 may also be integrated with the conformable portion 68 or optionally integrated with the support frame 62 itself as shown.

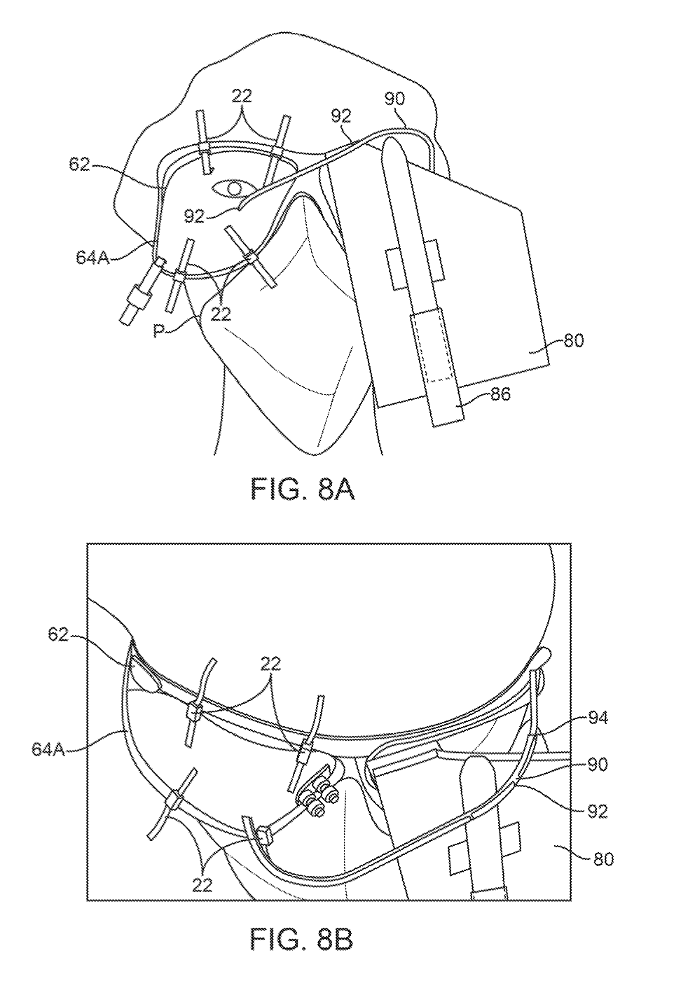

FIGS. 8A and 8B show front and perspective views, respectively, of yet another variation of a supporting frame 62 which may rest upon the patient's face and which may further support integrated platform 80, shown with an exemplary instrument 86 attached. In this variation, in addition to the platform 80 attached to the supporting frame 62, an optional light source 90 having an adjustable arm may also be attached to the supporting frame 62, e.g., via attachment 94. The light source 90 may be attached, for instance, along second supporting rim 64B and extend via an adjustable arm such that its distal end is in proximity to the first supporting rim 64A to provide additional lighting to the underlying eye to be treated.

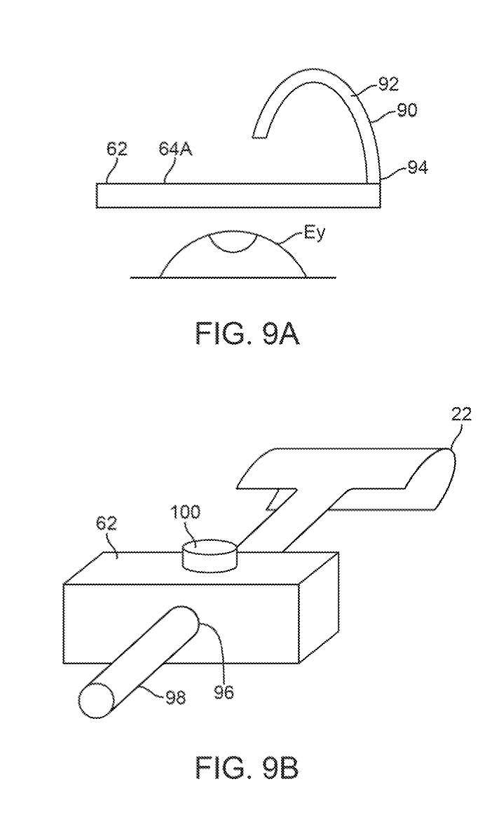

Additionally and/or alternatively, the light source 90 may incorporate a fluid lumen 92 through which irrigation fluid, such as saline, may be flowed to provide a clearing fluid in addition to lighting to the treated eye. FIG. 9A shows an exemplary illustration of how the light source 90 may be attached 94 along any portion of the supporting frame 62 provided that the distal end of the light source 90 (and/or fluid source) is in proximity to the treated eye EY.

FIG. 9B shows a detail perspective view of how the eyelid speculum 22 may be adjustably coupled to the supporting frame 62. In this example, the eyelid speculum 22 may be positioned at the end of a translatable member 98 which passes through a corresponding opening 96 through the supporting frame 62. An adjustable lock 100, such as a push button, may be integrated along the supporting frame 62 to releasably lock and unlock the translatable member 98 to provide for adjustable retraction of the eyelid.

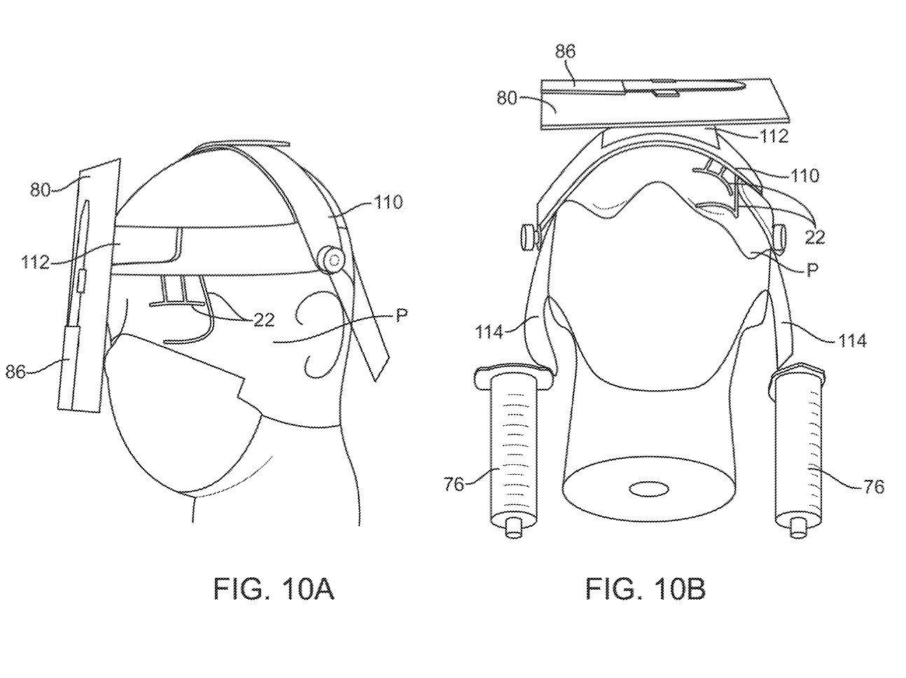

FIGS. 10A and 10B show various perspective views of yet another variation where an adjustable headband 110 positioned over the head of the patient P may provide a conforming support for the instrument platform 80. The platform 80 may be coupled to the headband 110 via platform attachment 112, such as over the forehead of the patient P, and one or more irrigation troughs 114 may be optionally attached or integrated with the headband 110 as well. Additionally, one or more eyelid speculums 22 may be adjustable coupled to extend from the headband 110 to be positioned over the treated eye to provide for surgical retraction of the eyelids.

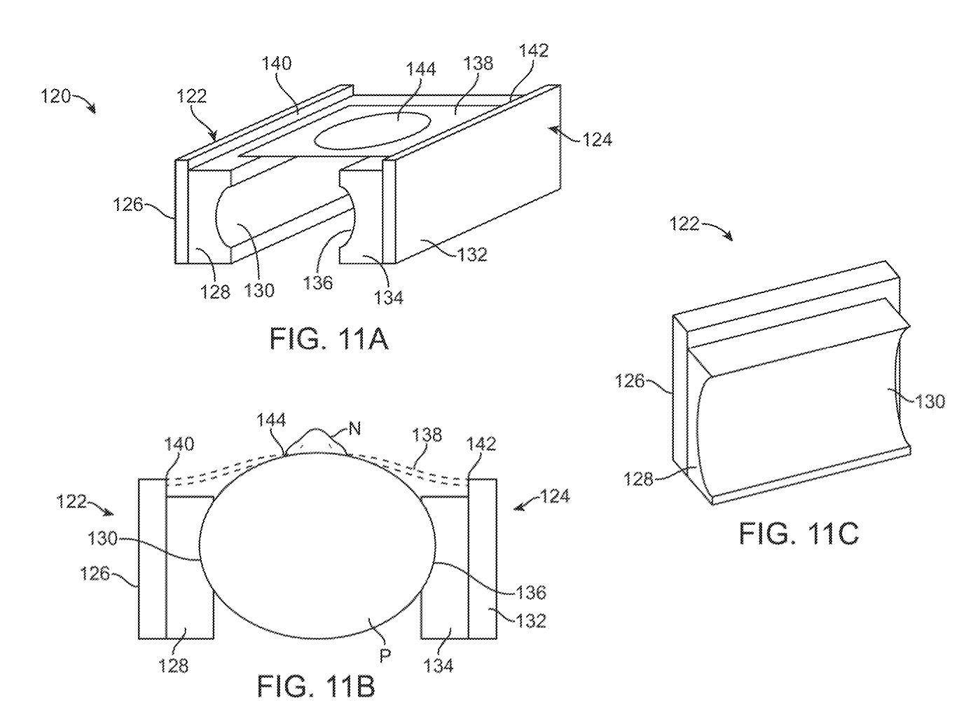

In yet another variation, FIGS. 11A and 11B show perspective and end views of an embodiment of an ophthalmic surgical block assembly 120 which may be positioned along either side of the patient's head to provide support and stabilization rather than being positioned to rest directly upon the patient's face. The surgical block assembly 120 may generally comprise a first block 122 and a second block 124 which may be positioned along both sides of the patient's head, as shown. The first block 122 may include a first backing 126 with a first conformable rest 128 which may be fabricated from any number of materials suitable for cushioning against the patient's head, such as foam. The conformable rest 128 may define a first contoured surface 130 which may be sized for placement against a side of the patient's head. A perspective view of the first block 122 with the first contoured surface 130 may be seen in FIG. 11C. Likewise, the second block 124 may include a second backing 132 and a second conformable rest 134 which defines a second contoured surface 136 for placement and cushioning against the other side of the patient's head in apposition to the first block 122.

With the patient's head secured between the first and second block 122, 124 which may be adjustably positioned relative to one another as well as relative to the patient's head, a conformable polymer shield 138 may be positioned over the patient's face to provide for draping of the patient. The shield 138 may be attached to the first and second block 122, 124 via respective first and second attachment 140, 142 and may further define an opening 144 through which the patient's face may be accessed. Alternatively, portions of the shield 138 may define one or more openings to accommodate one or both eyes for treatment as well as openings to accommodate the patient's nose and/or mouth. The size of each block 122, 124 may be optionally adjustable in height as well as relative spacing from one another and from the patient's head to accommodate varying head sizes, if so desired.

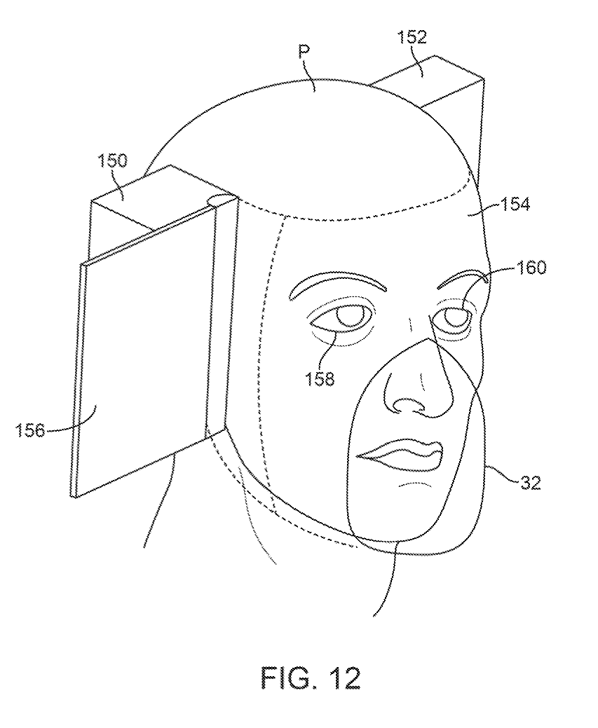

Another variation is illustrated in the perspective view of FIG. 12 which shows a first block 150 and second block 152 positioned on either side of the patient's head. The conformable polymer shield 154 is placed over the patient's face with a first opening 158 and a second opening 160 for providing access to the one or both of the patient's eyes for treatment. In this example, an optional oxygen mask 32 is also incorporated for placement over the patient's mouth and nose as well as a separate instrument rest 156 which may be positioned along one of both of the blocks 150, 152.

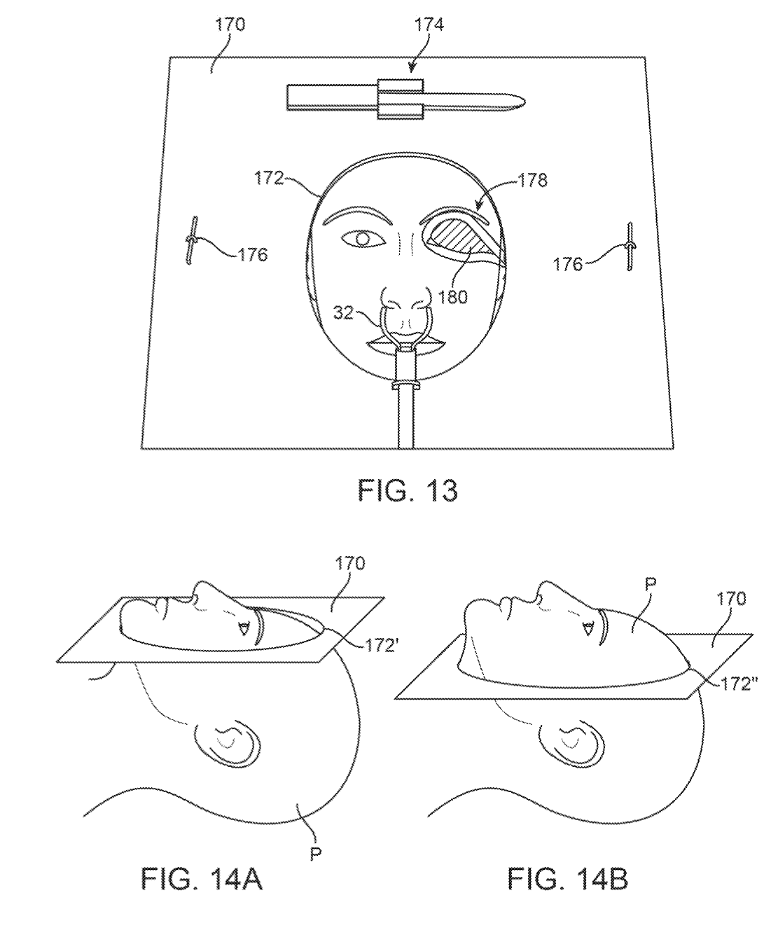

Yet another variation is illustrated in the perspective view of FIG. 13 which shows an example of a surgical platform 170 which may be placed around a patient's face. The platform 170 may provide a relatively broader surface upon which one or more instrument rests 174 may be provided. The platform 170 may be supported upon the patient's face or head and retained in position via a headband attached via one or more headband attachments 176 on either side of the platform 170. Moreover, the platform may define one or more openings 172 suitably sized for placement around a periphery of the patient's face in which case a conformable polymer shield 178 may be placed over the patient's face with one or more openings 180 for providing access to one or both eyes for treatment.

The opening 172 of the platform 170 for providing access to the patient's face may be varied in diameter as well for allowing for adjustability of the platform 170 relative to the patient's head. For instance, FIG. 14A shows one example where the opening of first diameter 172′ may be sized such that the platform 170 is positionable more ventrally upon the patient's face. FIG. 14B shows another example where the opening of second diameter 172″ may be sized relatively larger than first diameter 172′ to allow for the platform 170 to rest more dorsally upon the patient's face while provide further access to more of the patient's face as well as chin and/or forehead as well. In either case, the platform 170 may be supported directly by the patient and secured in place via a headband or the platform 170 may be supported by the patient alternatively via one or both blocks as described above.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A surgical platform assembly, comprising:
a support frame which is positionable upon a face of a patient, the support frame having at least a first supporting rim in proximity to at least one eye of the patient;
at least one speculum adjustably attached to the first supporting rim such that the at least one speculum is configured to retract at least an upper or lower eyelid of the patient;
a platform attached to the support frame and extending over at least a first part of the face of the patient; and
a conformable portion attached to the support frame adjacent to the platform and extending over a second part of the face of the patient.

2. The platform assembly of claim 1 wherein the support frame is comprised of an eyeglass-like frame.

3. The platform assembly of claim 1 wherein the support frame further comprises a second supporting rim in proximity to a remaining eye of the patient.

4. The platform assembly of claim 1 wherein the platform comprises a flattened surface having an instrument rest.

5. The platform assembly of claim 1 wherein the platform is attached to the support frame such that the platform extends over a forehead of the patient.

6. The platform assembly of claim 1 wherein the platform is attached to the support frame such that the platform extends over a remaining eye of the patient.

7. The platform assembly of claim 1 wherein the conformable portion is attached to the support frame such that the conformable portion extends over a remainder of the face of the patient.

8. The platform assembly of claim 1 wherein the conformable portion defines a contoured edge extending around a nose and mouth of the patient.

9. The platform assembly of claim 1 further comprising an oxygen mask positionable adjacent to the conformable portion.

10. The platform assembly of claim 1 further comprising an irrigation trough formed along a distal edge of the conformable portion.

11. The platform assembly of claim 1 further comprising an irrigation trough attached to the support frame.

12. A method of performing ophthalmic surgery, comprising:
providing a surgical platform assembly which is positionable upon a face of a patient, the assembly having a support frame which is positionable upon a face of a patient, the support frame having at least a first supporting rim in proximity to at least one eye of the patient, and at least one speculum adjustably attached to the first supporting rim such that the at least one speculum is configured to retract at least an upper or lower eyelid of the patient;
positioning the support frame upon at least a portion of the face of the patient such that the first supporting rim is positioned over the at least one eye; and,
retracting at least an upper or lower eyelid of the patient via the at least one speculum such that the upper or lower eyelid is retracted away from the patient via the first supporting rim.

13. The method of claim 12 wherein providing a surgical platform assembly further comprises providing a platform attached to the support frame and extending over at least a first part of the face of the patient.

14. The method of claim 13 further comprising providing a conformable portion attached to the support frame adjacent to the platform and extending over a second part of the face of the patient.

15. The method of claim 12 further comprising collecting an irrigation fluid within a trough formed integrally with the assembly.

16. The method of claim 12 further comprising providing an oxygen mask positioned adjacent to the assembly.

17. A surgical platform assembly, comprising:
a platform extending over at least a first part of a face of a patient, wherein the platform is securable upon a face of the patient;
a conformable portion attached to the platform and extending over a second part of the face of the patient, the conformable portion defining an opening for alignment with an eye of the patient to be treated; and,
at least one speculum adjustably attached to the conformable portion and in proximity to the opening such that the at least one speculum is configured to retract at least an upper or lower eyelid of the patient.

18. The assembly of claim 17 wherein the platform is securable upon the face of the patient via a headband.

19. The assembly of claim 17 wherein the conformable portion is comprised of a polymeric material.

20. The assembly of claim 17 wherein the conformable portion further comprises an irrigation trough along a distal edge of the conformable portion.

21. The assembly of claim 17 further comprising an irrigation trough defined along the platform.

22. The assembly of claim 17 wherein the platform further comprises an instrument rest upon the platform.

23. The assembly of claim 17 wherein the platform further comprises a handrest.

24. The assembly of claim 17 further comprising an oxygen mask positionable adjacent to the conformable portion.

25. A surgical platform assembly, comprising:
a headband adjustably securable over a head of a patient;
a platform attachable to the a portion of the headband;
at least one speculum adjustably attached to a second portion of the headband, where the at least one speculum extends from the second portion into proximity to at least one eye of the patient; and,
an irrigation trough fluidly coupled to the headband.

26. A surgical platform assembly, comprising:
a first block having a first contoured surface configured for placement against a first side of a patient's head;
a second block having a second contoured surface configured for placement against a second side of the patients head; and,
a conformable shield extending between the first block and second block for placement over a face of the patient's head, wherein the conformable shield defines at least one opening for alignment over at least one eye to be treated.

27. The assembly of claim 26 further comprising an oxygen mask positionable adjacent to the conformable shield.

28. The assembly of claim 26 further comprising a platform attached to the first block or second block.
